(19) United States
(12) Patent Application Publication
(10) Pub. No.: US 2026/0060568 A1
(43) Pub. Date: Mar. 5, 2026

(54) SYSTEM AND METHOD FOR MONITORING LUNG FUNCTION IN A SUBJECT WITH RESPIRATORY DYSFUNCTION

(71) Applicant: Monitored Therapeutics, Inc., Dublin, OH (US)

(72) Inventors: Alex Stenzler, Long Beach, CA (US); Mac Liaw, Westerville, OH (US); Martin Stegenga, Dublin, OH (US)

(21) Appl. No.: 19/265,183

(22) Filed: Jul. 10, 2025

Related U.S. Application Data
(60) Provisional application No. 63/687,377, filed on Aug. 27, 2024.

(51) Int. Cl. A61B 5/087; A61B 5/00

(52) U.S. Cl. CPC A61B 5/087; A61B 5/7271; A61B 5/742; A61B 5/746

(57) ABSTRACT

A method for monitoring lung function in a subject with respiratory dysfunction includes the steps of receiving a plurality of initial measurement data from a spirometry system indicative of a first plurality of respiratory efforts from the subject, calculating a first target value utilizing best-of-day data from the plurality of initial measurement data, receiving a plurality of subsequent measurement data from the spirometry system indicative of a second plurality of respiratory efforts from the subject, calculating a second target value utilizing best-of-day data from the plurality of subsequent measurement data, receiving a plurality of measurement data from the spirometry system indicative of a third plurality of respiratory efforts from the subject, and generating an alert if best-of-day data from the plurality of measurement data drops below a threshold set based on the second target value.

### CROSS-REFERENCE TO RELATED APPLICATIONS

This application claims priority to U.S. provisional application No. 63/687,377 filed Aug. 27, 2024, incorporated herein by reference in its entirety.

### BACKGROUND OF THE INVENTION

The remote monitoring of patients is only of value if the platform managing the monitoring is able to identify when a patient's condition is deteriorating and then notify someone that can review the event and determine if an action is required such as a medication change, or dose change, or a visit with their physician. With lung function in particular, since normal values vary significantly with a patient's age, height, gender and sometimes ethnicity, a universal fixed threshold would not be effective. And depending on the level of dysfunction as well as changes over time in the level of dysfunction, the threshold for an alert would not be the same as if they were normal. Further, if a single center is monitoring hundreds or more patients, the manual setting of target values for each patient would be an onerous process, particularly since they should track changes in each patient's condition over time, requiring updates perhaps every 30 days. Most sites do not set the initial target values or frequently update the target values.

Most sites conventionally use target values for alerts that are manually selected by multiple approaches.
(1) Provider sets absolute values for the alert thresholds.
(2) Provider select a drop (either percent or absolute change) from:
   (a) The baseline (first data) measurement.
   (b) The last measurement.
   (c) A trend (average) of the previous data. While this is actively updating the target, it looks at the mean value and not the higher value above the mean, which would represent the patient's best data in the period. It also is affected by values that are widely outside the mean that would artificially change the target based on the aberrant data.

Thus there is a need in the art for patient specific and dynamic algorithms used to identify alert thresholds as patient lung function changes. Embodiments described herein fit this need.

### SUMMARY OF THE INVENTION

In one embodiment, a method for treating a subject with respiratory dysfunction includes the steps of receiving of initial measurement data from a spirometry system indicative of respiratory efforts from the subject, calculating a first target value utilizing best-of-day data from the initial measurement data, receiving subsequent measurement data from the spirometry system indicative of a second set of respiratory efforts from the subject, calculating a second target value utilizing best-of-day data from the subsequent measurement data, receiving measurement data from the spirometry system indicative of a third set of respiratory efforts from the subject, and generating an alert if best-of-day data from the measurement data drops below a threshold correlated to the second target value. In one embodiment, the initial measurement data includes at least seven measurements taken at different times. In one embodiment, the best-of-day data is based on a set of predetermined standards. In one embodiment, the best-of-day data includes a first measurement parameter from a first respiratory effort and a second measurement parameter from a second respiratory effort, where the first and second respiratory efforts are performed at different times. In one embodiment, the first target value is calculated after adding one standard deviation of the initial measurement data to the initial measurement data. In one embodiment, the second target value is calculated after adding one standard deviation of the subsequent measurement data to the subsequent measurement data or a max value. In one embodiment, the second target value is calculated at least seven days after the first target value. In one embodiment, the method includes calculating a max value as a best-of-day parameter plus a predetermined set value, and resetting the second target value to the max value when the max value is lower than an initial calculation of a second target value. In one embodiment, the first target value includes at least one of a FEV1 parameter, a FVC parameter, and a PEF parameter. In one embodiment, the second target value includes at least one of a FEV1 parameter, a FVC parameter, and a PEF parameter. In one embodiment, the best-of-day data includes at least one of a FEV1 parameter, a FVC parameter, and a PEF parameter. In one embodiment, the method includes generating a first set of graphical target data based on measurement data within a first percentage of a target value, generating a second set of graphical target data based on measurement data within a second percentage of the target value, and generating a third set of graphical target data based on measurement data falling within a third percentage of the target value. In one embodiment, the first percentage is higher than the second percentage, and the second percentage is higher than the third percentage. In one embodiment, the first, second, and third percentage are each graphically depicted in different colors.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a diagram of a spirometry system according to one embodiment.

FIG. 2 is a diagram of a computing environment and monitoring communication system according to one embodiment.

FIG. 3 is a flow chart of a method for monitoring a subject with respiratory disfunction according to one embodiment.

FIG. 4 is a depiction of graphical target band data according to one embodiment.

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in systems and methods of monitoring lung function in subjects with respiratory dysfunction. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, +5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range. This may also apply for example to number of days, volumes, target values, low thresholds, high thresholds, FEV1, FVC, PEF or FEF25-75.

Referring now in detail to the drawings, in which like reference numerals indicate like parts or elements throughout the several views, in various embodiments, presented herein is a system and method of monitoring lung function in subjects with respiratory dysfunction.

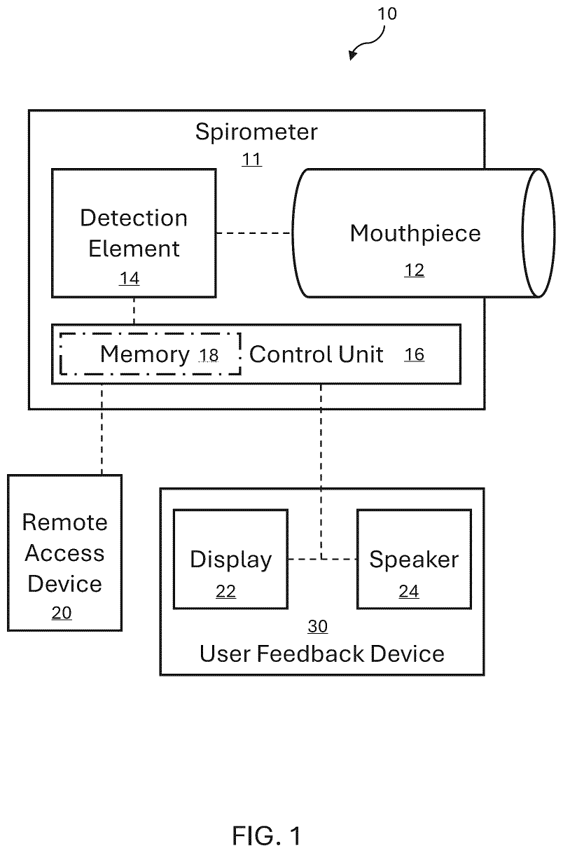

With reference to FIG. 1, the system and method of monitoring and treating subjects with respiratory dysfunction may include a spirometry system 10 having a spirometer 11 with a mouthpiece 12 opening for the patient to perform breathing maneuvers into. Generally, the patient takes a very deep breath and exhales as hard as possible for as long as possible into the mouthpiece 12. At least one detection element 14 is used to detect airflow rates from the patient. Different types of detection elements can be used to detect airflow. For instance, a transducer, such as an ultrasonic transducer can be used to generate an electrical measurement signal that measures the speed of airflow or a pressure difference in a channel. Other types of detection elements known in the art for spirometry, such as windmill-type mechanisms or a pneumotachometer can also be used to detect airflow characteristics and generate electrical signals indicative of the measurement. The detection element 14 is connected to a control unit 16, which processes a signal from the detection element 14 for calculating values such as volume and airflow rates. The control unit 16 can also include a memory 18 for storing software that executes the methods described herein. The memory 18 also stores measured values and calculated values so that the software can recall information from breathing maneuvers and utilize this information for making real-time or post-testing decisions on how to instruct the patient. In certain embodiments, the control unit 16 is integrated into the handheld spirometer 11. In other embodiments, the control unit 16 is part of a separate computing device, such as a laptop, tablet or smart phone, or it may alternatively be housed in user feedback device 30. It should be appreciated that the control unit 106 may be positioned in any type of computing device as would be understood by those skilled in the art, provided such computing device is connected to one or more detection elements 14 of spirometer 11 via a communications network. As contemplated herein, any spirometer, user feedback device or other computing device may generally include at least one processor, standard input and output devices, as well as all hardware and software typically found on computing devices for storing data and running programs, and for sending and receiving data over a network. Accordingly, control unit 16 may freely communicate with a remote access device 20. The remote access device 20 may be any type of computing device described herein and can be used by an off-site medical professional for setting certain parameters, thresholds and ranges disclosed herein. The control unit 16 can also send test results to the remote access device 20 so that results can be monitored and viewed by the off-site medical professional, even though the patient is able to complete the testing by themselves, entirely within the home care setting. The control unit 16 also communicates with a user feedback device 30 having at least a display 22 and speaker 24 to present audio or visual instructions, test results, or other types of data or information disclosed herein. As contemplated herein, user feedback device 30 may also be any sort of computing device described herein, including desktop or moble devices, laptops, tablets, wireless digital/cellular phones, smart phones, televisions or other thin client devices as would be understood by those skilled in the art. In certain embodiments, a component of the user feedback device, such as the speaker 24, may be integrated into the spirometer 11. System preferences and other types of information can be sent to the control unit 16 through an input interface integrated with or connected to the user feedback device 30 such as a keyboard, touchscreen or voice command module.

Without limitation, other additional computing devices may be used with the system, including desktop or moble devices, laptops, tablets, wireless digital/cellular phones, televisions or other thin client devices as would be understood by those skilled in the art. Further, the system 10 has associated therewith a software platform that may operate as a local or remote executable software platform. For example, the computer operable component(s) of the system may reside entirely on a single computing device, or may reside on any number of devices within the system. Similar to control unit 16, remote access device 20 and user feedback device 30, any computing devices contemplated herein may generally include at least one processor, standard input and output devices, as well as all hardware and software typically found on computing devices for storing data and running programs, and for sending and receiving data over a network. Any computing device forming part of the system 10 may also be connected directly or via a network to remote databases, such as for additional storage backup, and to allow for the communication of files, email, software, and any other data format between two or more computing devices. There are no limitations to the number, type or connectivity of the databases utilized by the system of the present invention.

The system 10 may include a communications network as would be understood by those having ordinary skill in the art, such as, for example, an open, wide area network (e.g., the internet), an electronic network, an optical network, a wired or wireless network, a physically secure network or virtual private network, connection and any combinations thereof. The communications network may also include any intermediate nodes, such as gateways, routers, bridges, internet service provider networks, public-switched telephone networks, proxy servers, firewalls, and the like, such that the communications network may be suitable for the transmission of information items and other data throughout the system 10.

Further, the communications network may use standard architecture and protocols as understood by those skilled in the art, such as, for example, a packet switched network for transporting information and packets in accordance with a standard transmission control protocol/Internet protocol (“TCP/IP”). Any of the computing devices may be communicatively connected into the communications network through, for example, a traditional telephone service connection using a conventional modem, an integrated services digital network (“ISDN”), a cable connection including a data over cable system interface specification (“DOCSIS”) cable modem, a digital subscriber line (“DSL”), a T1 line, or any other mechanism as understood by those skilled in the art. Additionally, the system may utilize any conventional operating platform or combination of platforms (Windows, Mac OS, Unix, Linux, Android, etc.) and may utilize any conventional networking and communications software as would be understood by those skilled in the art.

To protect data, an encryption standard may be used to protect files from unauthorized interception over the network. Any encryption standard or authentication method as may be understood by those having ordinary skill in the art may be used at any point in the system of the present invention. For example, encryption may be accomplished by encrypting an output file by using a Secure Socket Layer (SSL) with dual key encryption. Additionally, the system may limit data manipulation, or information access.

As mentioned previously, the system may include application software, which may be managed by a local or remote computing device. The software may include a software framework or architecture that optimizes ease of use of at least one existing software platform, and that may also extend the capabilities of at least one existing software platform. The application architecture may approximate the actual way users organize and manage electronic files, and thus may organize use activities in a natural, coherent manner while delivering use activities through a simple, consistent, and intuitive interface within each application and across applications. The architecture may also be reusable, providing plug-in capability to any number of applications, without extensive re-programming, which may enable parties outside of the system to create components that plug into the architecture. Thus, software or portals in the architecture may be extensible and new software or portals may be created for the architecture by any party.

The system may provide software accessible to one or more users to perform one or more functions. Such applications may be available at the same location as the user, or at a location remote from the user. Each application may provide a graphical user interface (GUI) for ease of interaction by the user with information resident in the system. A GUI may be specific to a user, set of users, or type of user, or may be the same for all users or a selected subset of users. The system software may also provide a master GUI set that allows a user to select or interact with GUIs of one or more other applications, or that allows a user to simultaneously access a variety of information otherwise available through any portion of the system.

The system software may also be a portal or SaaS that provides, via the GUI, remote access to and from the system of the present invention. The software may include, for example, a network browser, as well as other standard applications. The software may also include the ability, either automatically based upon a user request in another application, or by a user request, to search, or otherwise retrieve particular data from one or more remote points, such as on the internet or from a limited or restricted database. The software may vary by user type or may be available to only a certain user type, depending on the needs of the system. Users may have some portions, or all of the application software resident on a local computing device, or may simply have linking mechanisms, as understood by those skilled in the art, to link a computing device to the software running on a central server via the communications network, for example. As such, any device having, or having access to, the software may be capable of uploading, or downloading, any information item or data collection item, or informational files to be associated with such files.

Presentation of data through the software may be in any sort and number of selectable formats. For example, a multi-layer format may be used, wherein additional information is available by viewing successively lower layers of presented information. Such layers may be made available by the use of drop-down menus, tabbed folder files, or other layering techniques understood by those skilled in the art or through a novel natural language interface as described herein. All formats may be in standard readable formats, such as XML. The software may further incorporate standard features typically found in applications, such as, for example, a front or “main” page to present a user with various selectable options for use or organization of information item collection fields.

The system software may also include standard reporting mechanisms, such as generating a printable results report, or an electronic results report that can be transmitted to any communicatively connected computing device, such as a generated email message or file attachment. Likewise, particular results of the aforementioned system can trigger an alert signal, such as the generation of an alert email, text or phone call, to alert a user of the particular results. Further embodiments of such mechanisms are described elsewhere herein or may standard systems understood by those skilled in the art.

A system architecture will be described as an example embodiment for implementing a method for treating a patient with respiratory illness. Embodiments of the method for treating a patient with respiratory dysfunction will at times involve one or more spirometry devices and computing devices accessed by the patient, a patient caregiver, a specialist, and medical support staff. In some aspects of the present invention, software executing the instructions provided herein may be stored on a non-transitory computer-readable medium, wherein the software performs some or all of the steps of the method when executed on a processor.

Aspects of the method for treating a patient with respiratory dysfunction relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the method for treating a patient with respiratory dysfunction may be executed on any acceptable computing platform, including but not limited to a server, a cloud instance, a workstation, a thin client, a mobile device, an embedded microcontroller, a television, or any other suitable computing device known in the art.

Parts of the method for treating a patient with respiratory illness are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of the method for treating a patient with respiratory illness are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

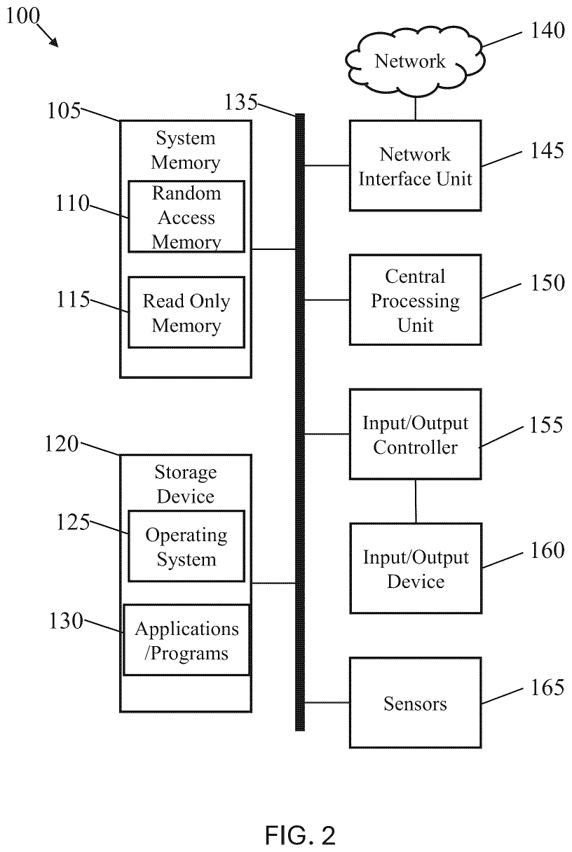

FIG. 2 and the following discussion are intended to provide a brief, general description of a suitable computing environment and communication system in which the method for treating a patient with respiratory dysfunction may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hospital or medical facility computer systems, hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 2 depicts an illustrative computer architecture for a computer 100 for practicing the various embodiments of the method for treating a patient with respiratory illness. The computer architecture shown in FIG. 2 illustrates a conventional computer system, including a central processing unit 150 (“CPU”), a system memory 105, including a random-access memory 110 (“RAM”) and a read-only memory (“ROM”) 115, and a system bus 135 that couples the system memory 105 to the CPU 150. A basic input/output system containing the basic routines that help to transfer information between elements within the computer, such as during startup, is stored in the ROM 115. The computer 100 further includes a storage device 120 for storing an operating system 125, application/program 130, and data.

The storage device 120 is connected to the CPU 150 through a storage controller (not shown) connected to the bus 135. The storage device 120 and its associated computer-readable media provide non-volatile storage for the computer 100. Although the description of computer-readable media contained herein refers to a storage device, such as a hard disk or CD-ROM drive, it should be appreciated by those skilled in the art that computer-readable media can be any available media that can be accessed by the computer 100.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the method for treating a patient with respiratory illness, the computer 100 may operate in a networked environment using logical connections to remote computers through a network 140, such as TCP/IP network such as the Internet or an intranet. The computer 100 may connect to the network 140 through a network interface unit 145 connected to the bus 135. It should be appreciated that the network interface unit 145 may also be utilized to connect to other types of networks and remote computer systems.

The computer 100 may also include an input/output controller 155 for receiving and processing input from a number of input/output devices 160, including a keyboard, a mouse, a touchscreen, a camera, a microphone, a controller, a joystick, or other type of input device. Similarly, the input/output controller 155 may provide output to a display screen, a printer, a speaker, or other type of output device. The computer 100 can connect to the input/output device 160 via a wired connection including, but not limited to, fiber optic, Ethernet, or copper wire or wireless means including, but not limited to, Wi-Fi, Bluetooth, Near-Field Communication (NFC), infrared, or other suitable wired or wireless connections.

As mentioned briefly above, a number of program modules and data files may be stored in the storage device 120 and/or RAM 110 of the computer 100, including an operating system 125 suitable for controlling the operation of a networked computer. The storage device 120 and RAM 110 may also store one or more applications/programs 130. In particular, the storage device 120 and RAM 110 may store an application/program 130 for providing a variety of functionalities to a user. For instance, the application/program 130 may comprise many types of programs such as a word processing application, a spreadsheet application, a desktop publishing application, a database application, a gaming application, internet browsing application, electronic mail application, messaging application, and the like. According to an embodiment of the present invention, the application/program 130 comprises a multiple functionality software application for providing word processing functionality, slide presentation functionality, spreadsheet functionality, database functionality and the like.

The computer 100 in some embodiments can include a variety of sensors 165 for monitoring the environment surrounding and the environment internal to the computer 100. The sensors may include measurements from the detection element 14 of the spirometer 11. The sensors 165 may include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor. Physiological sensors such as pulse oximeters to measure the oxygen saturation levels in blood and heart rate monitors may be utilized. Other physiological sensors may include but are not limited to weight scales, weight scales with bioimpedance measurements of fluid, or devices for measuring hand grip strength, exhaled nitric oxide, forced oscillation impedance, temperature, and activity.

Embodiments of the system have the ability to set lung function target values for patients, which are used to trigger alerts should the patient's lung function drop below specified thresholds. Embodiments of the system are able to turn automatic targeting adjustments on or off for each site, enabling self-control of target values for sites that prefer to adjust them manually on their own.

Automatically adjusting target values for patients is an important capability of the system so that health care providers can be notified when a patent's lung function has a clinically important change. Embodiments can also visually identify graphical bands where the top of the band is the set target value, and the bottom of the band is at 90% of the target value. Embodiments may include markers for when the patient's data drops below 80% as well. The dynamic setting of target values ensures providers are able to appropriately follow alterations in lung function as they change. If a patient's lung function is improving either due to better disease management or as typically occurs following a lung transplant and if the target is not adjusted upwards and tracking their improvements, then the platform would never issue an alert should the patient's lung function take a downward turn until the patient returned back to their initial status.

Another advantage is situations when the patient's lung function is deteriorating over time due to the progression of their disease and it is appropriate to lower the target so that the alerts are not issued following every measurement when compared to the initial target value and are meaningful to the provider managing their disease.

The setting of the target values is based on the analysis of Best-of-Day measurements. When a patient goes to a hospital PFT laboratory, the patient may perform up to 8 attempts. However, the report generated for the physician to interpret are the best values and not a mean value. Embodiments of the algorithm described herein work on the same principle for data collected from a patient at home. The Best-of-Day measurement process selects the best data as defined by predetermined standards (e.g. the ATS/ERS 2019 Spirometry standards) from all measurements performed on a single day whether the patient performed a single measurement or 8 measurements. This also means that the Best FEV1 and Best FVC may not come from the same measurement, etc.

In one embodiment, the updated target value is automatically set every 30 days, initially using the last 7 measurement days of Best-of-Day spirometry (usually reflects multiple weeks of measurements). To account for slight variability in this effort dependent measurement, using the highest value may bias the data towards more alarms, particularly if there is a large variability in the patient's measurements. Therefore, the target value is determined to be the 7 measurement day Mean value plus 1 Standard Deviation (SD) for the measurement period. One standard deviation represents the highest value of 95% of the last 7 measurement days data within that 30-day period. To account for patients who have day to day values far outside the target values (resulting from poor effort, technical issues, etc.) where that high standard deviation might create aberrant target values, the algorithm creates a user settable maximum allowed target above the mean as a guardrail. Therefore, target values are the Mean plus 1 SD or the “Max Value” (see an example embodiment below) for each parameter, whichever is lower.
FEV1: Mean plus 1 SD or “Max Value: (“Max Value”=Mean plus 200 mL).
FVC: Mean plus 1 SD or “Max Value: (“Max Value”=Mean plus 200 mL).
PEF: Mean plus 1 SD or “Max Value: (“Max Value”=Mean plus 1 L/sec).
FEF25-75: Mean plus 1 SD or “Max Value: (“Max Value”=Mean plus 1 L/sec).

For example, if the mean FVC is 4.00 L and the SD is 300 mL, then the target would be 4.20 L or 200 mL above the mean and not 300 mL above the mean.

As long as the Mean values are increasing, the target values will be updated upwards after each 30-day calculation from the last 7 measurement days. If a 7 measurement day Mean value is decreasing, the algorithm switches and uses the last 60-days of data to calculate the 60-day Mean for calculating alternative target values, but using the higher of the two as the final target. This protects the algorithm from missing alerts in patients whose lung function is deteriorating if it also rapidly lowered the target values. If after 60 days the Mean is stabilized at a lower value, the target will be appropriately set. If it continues to decline, the target will always be decreased slower than the patient's decline so that alerts are still always functional.

The target values are determined individually for each parameter. So, for example, a patient whose FEV1 is decreasing while the FVC increases, will have the FVC target adjusted upwards based on the last 7 measurement-day data, while the FEV1 target would be adjusted downwards using the 60-day data. In certain embodiments, the 60-day analysis may alternatively be a longer or shorter day analysis (e.g. 45 days, 75 days, 90 days, etc.), an analysis based on number of measurements (e.g. 15 measurements, 20 measurements, 25 measurements, etc.), or based on whichever day or measurement metric comes first.

The building of the target values for a patient begins following a structure of the algorithm depending on where the patient currently is on the system according to one embodiment:
(1) Patient with no existing data, starting from zero:
   (a) Collect data for the first 7 measurement days and calculate target value.
   (b) After there are 7 measurement days, every 30 calendar days thereafter, use the best-of-day data from the last 7 measurement days to calculate a new target.
(2) Patient with at least 7 measurement days of data:
   (a) Use the best of the last 7 measurement days to calculate a new target.
   (b) Every 30 calendar days thereafter, use the best-of-day data from the last 7 measurement days to calculate a new target.

The target values are then updated every 30 calendar days after the last change. If manual changes are made mid-month, the algorithm restarts the 30-day window to evaluate for a change again. There are different approaches for when the target values are increasing versus when they are decreasing.

The system is able to turn automatic targeting adjustments on or off for each site, enabling self-control of target values for sites that prefer to adjust them manually on their own.

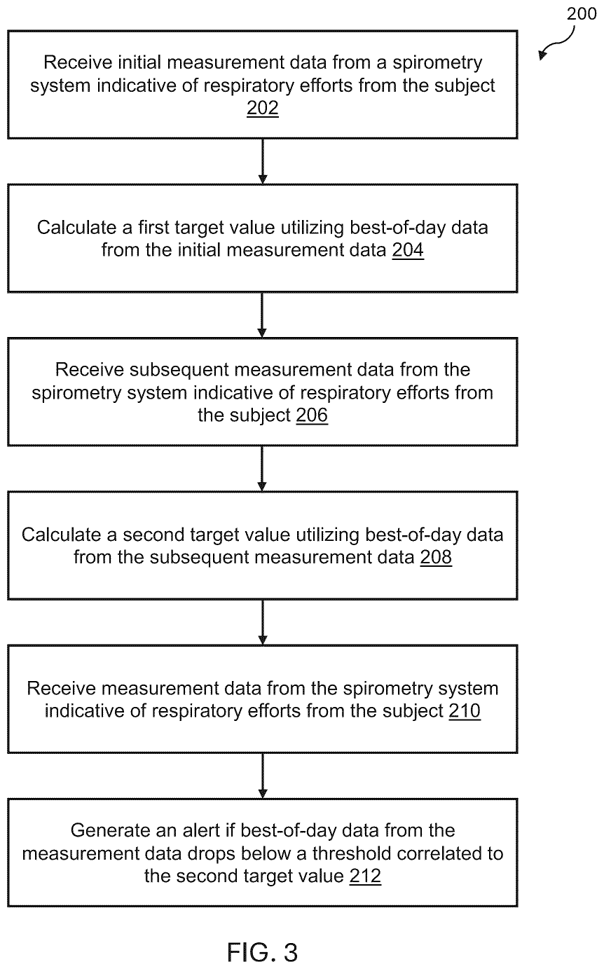

Accordingly, with reference now to FIG. 3, a method 200 for treating a subject with respiratory dysfunction is shown according to one embodiment. The method 200 includes the steps of receiving a set of initial measurement data from a spirometry system indicative of a set of respiratory efforts from the subject 202, calculating a first target value utilizing best-of-day data from the set of initial measurement data 204, receiving a set of subsequent measurement data from the spirometry system indicative of a second set of respiratory efforts from the subject 206, calculating a second target value utilizing best-of-day data from the set of subsequent measurement data 208, receiving a set of measurement data from the spirometry system indicative of a third set of respiratory efforts from the subject 210, and generating an alert if best-of-day data from the set of measurement data drops below a threshold correlated to the second target value 212. In one embodiment, the set of initial measurement data comprises at least seven measurements taken at different times. As discussed above, in one embodiment, the best-of-day data is based on a set of predetermined standards. In one embodiment, the best-of-day data includes a first measurement parameter from a first respiratory effort and a second measurement parameter from a second respiratory effort, wherein the first and second respiratory efforts are performed at different times. In one embodiment, the first target value is calculated after adding one standard deviation of the set of initial measurement data to the set of initial measurement data. In one embodiment, the second target value is calculated after adding one standard deviation of the set of subsequent measurement data to the set of subsequent measurement data or a max value. In one embodiment, the second target value is calculated at least seven days after the first target value. In one embodiment, the method includes the steps of calculating a max value as a best-of-day parameter plus a predetermined set value and resetting the second target value to the max value when the max value is lower than an initial calculation of a second target value. In one embodiment, the first target value includes at least one of a FEV1 parameter, a FVC parameter, and a PEF parameter. In one embodiment, the second target value includes at least one of a FEV1 parameter, a FVC parameter, and a PEF parameter. In one embodiment, the best-of-day data includes at least one of a FEV1 parameter, a FVC parameter, and a PEF parameter.

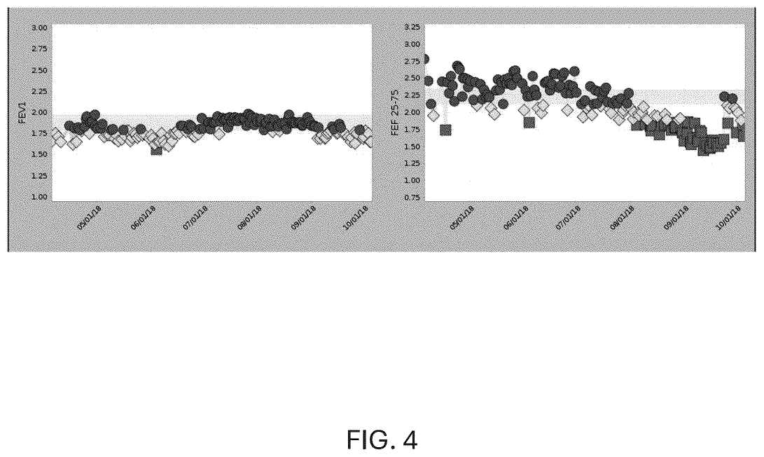

In one embodiment, target bands are generated on the system for transition from green to yellow to red based on a percentage of the target value (see e.g. FIG. 4). The bands for the high threshold (transition from green to yellow) for FEV1, FVC, and FEF25-75 are in one embodiment set at ≤90% of the target value and low threshold (transition from yellow to red) is set at ≤80% of the target value. For PEF, green to yellow is set at ≤80% and low threshold (transition from yellow to red) is set at ≤50%. Note that the lower thresholds are reflected in the color of the data points on the graph and not in additional bands on the screen.

Examples of calculation of bands for FEV1:
(1) FEV1 mean 1.50, SD of 120 mL where Target becomes 1.62
Yellow band begins at 0.90*1.62 L=1.458 L
Red band begins at 0.80*1.62 L=1.296 L
(2) FEV1 mean 4.00, SD of 140 mL where Target becomes 4.14
Yellow band begins at 0.9*4.14 L=3.726 L
Red band begins at 0.8*4.14 L=3.312 L

Accordingly, in one embodiment, a method for treating a subject with respiratory dysfunction may further include the steps of generating a first set of graphical target data based on measurement data within a first percentage of a target value, generating a second set of graphical target data based on measurement data within a second percentage of the target value, and generating a third set of graphical target data based on measurement data falling within a third percentage of the target value. In one embodiment, the first percentage is higher than the second percentage, and the second percentage is higher than the third percentage. In one embodiment, the first, second, and third percentage are each graphically depicted in different colors.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A method for monitoring lung function in a subject with respiratory dysfunction comprising:
receiving a plurality of initial measurement data from a spirometry system indicative of a first plurality of respiratory efforts from the subject;
calculating a first target value utilizing best-of-day data from the plurality of initial measurement data;
receiving a plurality of subsequent measurement data from the spirometry system indicative of a second plurality of respiratory efforts from the subject;
calculating a second target value utilizing best-of-day data from the plurality of subsequent measurement data;
receiving a plurality of measurement data from the spirometry system indicative of a third plurality of respiratory efforts from the subject; and
generating an alert if best-of-day data from the plurality of measurement data drops below a threshold correlated to the second target value.

2. The method of claim 1, wherein the plurality of initial measurement data comprises at least seven measurements taken at different times.

3. The method of claim 1, wherein the best-of-day data is based on a set of predetermined standards.

4. The method of claim 1, wherein the best-of-day data includes a first measurement parameter from a first respiratory effort and a second measurement parameter from a second respiratory effort, wherein the first and second respiratory efforts are performed at different times.

5. The method of claim 1, wherein the first target value is calculated after adding one standard deviation of the plurality of initial measurement data to the plurality of initial measurement data.

6. The method of claim 1, wherein the second target value is calculated after adding one standard deviation of the plurality of subsequent measurement data to the plurality of subsequent measurement data or a max value.

7. The method of claim 1, wherein the second target value is calculated at least seven days after the first target value.

8. The method of claim 1 further comprising:
calculating a max value as a best-of-day parameter plus a predetermined set value; and
resetting the second target value to the max value when the max value is lower than an initial calculation of a second target value.

9. The method of claim 1, wherein the first target value includes at least one of a FEV1 parameter, a FVC parameter, and a PEF parameter.

10. The method of claim 1, wherein the second target value includes at least one of a FEV1 parameter, a FVC parameter, and a PEF parameter.

11. The method of claim 1, wherein the best-of-day data includes at least one of a FEV1 parameter, a FVC parameter, and a PEF parameter.

12. The method of claim 1 further comprising:
generating a first set of graphical target data based on measurement data within a first percentage of a target value;
generating a second set of graphical target data based on measurement data within a second percentage of the target value; and
generating a third set of graphical target data based on measurement data falling within a third percentage of the target value.

13. The method of claim 12, wherein the first percentage is higher than the second percentage, and the second percentage is higher than the third percentage.

14. The method of claim 1, wherein the first, second, and third percentage are each graphically depicted in different colors.
